(19) United States
(12) Patent Application Publication
(10) Pub. No.: US 2026/0301913 A1
(43) Pub. Date: Oct. 1, 2026

(54) IDENTIFICATION OF ARTERIAL DISEASE PATIENTS FOR FOLLOW-UP

(71) Applicant: Medtronic Vascular, Inc., Santa Rosa, CA (US)

(72) Inventor: Paul J. Coates, Corte Madera, CA (US)

(21) Appl. No.: 19/480,589

(22) PCT Filed: Apr. 18, 2024
(86) PCT No.: PCT/US2024/025196

§ 371 Date: Oct. 31, 2025

Related U.S. Application Data
(60) Provisional application No. 63/500,376, filed on May 5, 2023.

(51) Int. Cl. G16H 20/40; G16H 30/40; G16H 50/20; G16H 50/30; G16H 50/50

(52) U.S. Cl. CPC G16H 20/40; G16H 30/40; G16H 50/20; G16H 50/30; G16H 50/50

(57) ABSTRACT

Example medical systems and techniques are disclosed. An example medical system includes processing circuitry coupled to memory. The processing circuitry is configured to determine a culprit lesion based on a computational model of vasculature of a patient. The processing circuitry is configured to virtually remove the culprit lesion from the computational model to generate a revised computational model. The processing circuitry is configured to determine a plurality of shear stresses along arterial walls in an arterial tree based on the revised computational model and determine that at least one of the plurality of shear stresses meets at least one threshold in at least one location within the arterial tree. The processing circuitry is configured to, based on the at least one of the plurality of shear stresses meeting the at least the threshold, identify the patient as a member of a first patient group for follow-up.

This application claims the benefit of priority from U.S. Provisional Patent Application No. 63/500,376, filed May 5, 2023, the entire contents of which are incorporated herein by reference.

### TECHNICAL FIELD

This disclosure relates to identifying arterial disease patients for additional follow-up treatment or monitoring.

### BACKGROUND

Patients having vascular issue(s), such as coronary arterial disease, may have varying disease severity and varying issues. Surgery on such patients to treat the vascular issue(s) may cause other vascular issue(s) to appear. For example, removal or bypass of a lesion in one portion of the vasculature of a patient may cause or reveal additional issue(s) within the vasculature of that patient.

### SUMMARY

Medical conditions, such as coronary arterial disease, may develop over years or even decades. As coronary arterial disease develops and modifies blood flow, the arterial bed may remodel to be optimal for the current blood flow conditions. For example, the arterial tree may remodel to maintain wall shear stress within an optimal range.

At some time, the arterial disease may reach a point that requires medical intervention. At the time of medical intervention, the disease is treated and years of atherosclerotic impact are reversed immediately, or almost immediately. However, the new flow conditions are not consistent with the years of remodeling that have occurred. This remodeling process may be a relatively slow process that may not be rapid enough to quickly catch up with the change in flow conditions caused by a medical intervention. As such, a subset of patients being treated for coronary arterial disease may develop new disease associated with a mis-match between the new blood flow and the remodeled arterial tree.

It is becoming more common to develop a computational model of the heart, for example, pre-procedurally using computed tomography (CT) data, or peri-procedural data using fluoroscopic data. Such techniques have been successful in identifying culprit lesions by calculating fractional flow reserve (FFR) values or other metrics of the disruption to the ideal or maximal flow caused by the presence of a culprit lesion.

Because patients having vascular issue(s), such as coronary arterial disease, may have varying disease severity and varying issues, patients may have different levels of need for treatment. For example, one patient with coronary arterial disease may be treated to remove or bypass a lesion. Such treatment on the patient with coronary arterial disease may reveal other issues in the patient due to the mismatch of the new blood flow and the remodeled arterial tree. While a second patient treated in the same general manner, may not experience the other issues which the first patient experienced. As such, it may be desirable to identify and distinguish which patients may require further, more urgent treatment than those patients who do not, so as to more efficiently utilize medical resources on the patients requiring more urgent treatment. Additionally, in some examples, it may be desirable to make such an identification during a diagnostic procedure, prior to any medical intervention. By doing so, one may improve patient outcomes, particularly for those patients who may require more medical attention than other patients.

In one example, the disclosure describes a medical system comprising memory configured to store a first computational model of a first patient vasculature; and processing circuitry communicatively coupled to the memory, the processing circuitry being configured to: determine a first culprit lesion based on the first computational model of the vasculature of the first patient; virtually remove the first culprit lesion from the first computational model to generate a revised first computational model; determine a first plurality of shear stresses along first arterial walls in a first arterial tree based on the revised first computational model; determine that at least one of the first shear stresses meets at least one threshold in at least one location within the first arterial tree; and based on the at least one of the first plurality of shear stresses meeting the at least one threshold, identify the patient as a member of a first patient group for follow-up treatment.

In another example, the disclosure describes a method comprising: determining, by processing circuitry, a first culprit lesion based on a first computational model of vasculature of a first patient; virtually removing, by the processing circuitry, the first culprit lesion from the first computational model to generate a revised first computational model; determining, by the processing circuitry, a first plurality of shear stresses along first arterial walls in a first arterial tree based on the revised first computational model; determining, by the processing circuitry, that at least one of the first plurality of shear stresses meets at least one threshold in at least one location within the first arterial tree; and based on the at least one of the first plurality of shear stresses meeting the at least one threshold, identifying, by the processing circuitry, the patient as a member of a first patient group for follow-up treatment.

In yet another example, the disclosure describes a non-transitory computer readable medium comprising instructions, which, when executed, cause processing circuitry to determine a first culprit lesion based on a first computational model of vasculature of a first patient; virtually remove the first culprit lesion from the first computational model to generate a revised first computational model; determine a first plurality of shear stresses along first arterial walls in a first arterial tree based on the revised first computational model; determine that at least one of the first plurality of shear stresses meets at least one threshold in at least one location within the first arterial tree; and based on the at least one of the first plurality of shear stresses meeting the at least one threshold, identify the patient as a member of a first patient group for follow-up treatment.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for stratifying patients according to one or more aspects of this disclosure.

FIG. 2 is a schematic view of one example of a computing system of the system of FIG. 1.

FIG. 3 is a flow diagram of example techniques for identifying patients for follow-up according to one or more aspects of this disclosure.

FIG. 4 is a conceptual diagram illustrating an example machine learning model according to one or more aspects of this disclosure.

FIG. 5 is a conceptual diagram illustrating an example training process for a machine learning model according to one or more aspects of this disclosure.

### DETAILED DESCRIPTION

Medical resources are typically limited. To most efficiently use the limited medical resources, patients requiring more medical attention may be prioritized over patients requiring less medical attention. As such, it may be desirable to identify those patients requiring more medical attention and prioritize them for further medical treatment over patients not requiring more, or requiring less, medical attention. Doing so may improve patient outcomes and conserve medical resources, such as clinician time, hospital beds, equipment used during procedures, or the like.

A medical system may determine a culprit lesion in vasculature of a patient, for example, based on FFR values. The medical system may virtually remove the culprit lesion and recompute the fluid dynamics calculation to determine new velocity contours throughout an arterial tree. The medical system may also determine the shear stress along the arterial walls (e.g., using the velocity gradient at the vessel wall). The medical system may determine a subset of patients where the new shear stress is no longer within an optimal range at one or more places in the arterial tree. Additionally, or alternatively, the system may determine a further subset of patients where the shear stress is not in the optimal range at specific locations, for example, at or near a carina, or a side-branch bifurcation etc.

The system may stratify the sub-groups described above (shear stress outside normal range) as high risk and identify those patients to receive a more intensive follow-up protocol over time, such as incremental stress tests, CT scans, or more invasive testing, such as angiography. Other sub-groups where the new shear stress is considered to be within an optimal range might be deprioritized from an extensive follow-up and treated with less intensive follow up. In some examples, the stratification may occur in real-time while a patient is undergoing a medical procedure. In some examples, the stratification may occur after the medical procedure is complete, for example, hours, days, weeks, or the like, after the medical procedure is complete.

Aspects of this disclosure are applicable to at least Catheterization lab (Cath lab) procedures. Example Cath lab procedures include, but are not necessarily limited to, coronary diagnostic procedures, coronary intervention procedures (e.g., treatments for ST elevation myocardial infarction (STEMI), treatments for chronic total occlusion (CTO), coronary artery bypass graft (CABG), or the like), renal denervation (RDN) procedures, structural heart and aortic (SH&A) procedures (e.g., transcatheter aortic valve replacement (TAVR), transcatheter mitral valve replacement (TMVR), and the like), device implantation procedures (e.g., heart monitors, pacemakers, defibrillators, and the like), etc.

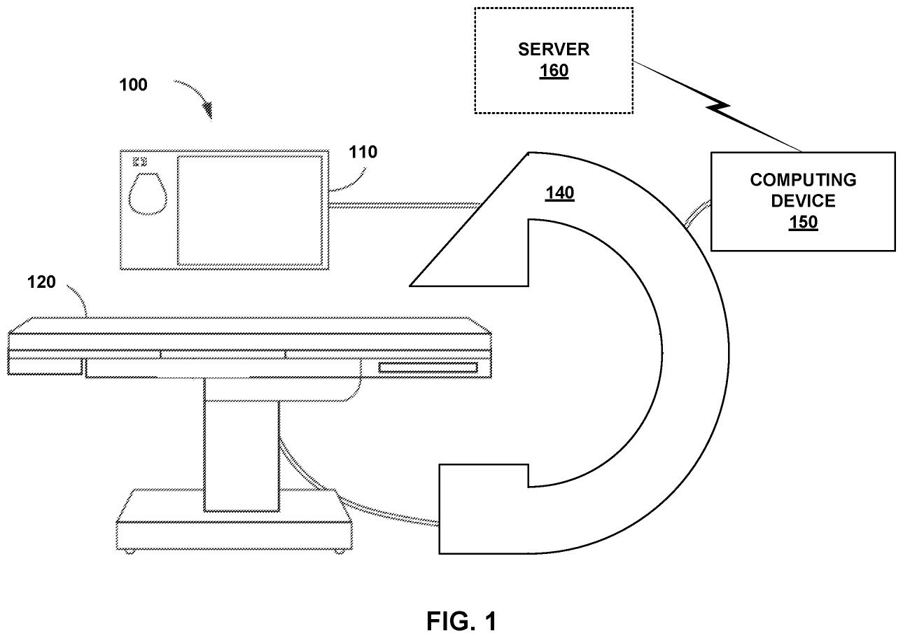

FIG. 1 is a schematic perspective view of one example of a system for stratifying patients according to one or more aspects of this disclosure. System 100 includes a display device 110, a table 120, an imager 140, and a computing device 150. System 100 may be an example of a system for use in an emergency room or a Cath lab. In some examples, system 100 may include other devices, not shown for simplicity purposes. In some examples, system 100 may also include server 160, which may be co-located with the other devices of system 100 or may be located elsewhere. System 100 may be used during a medical procedure, such as an emergency medical procedure to treat a medical condition of a patient. During such a procedure, system 100 may identify a patient as a member of a first patient group for follow-up treatment or a second patient group. The first patient group may have a higher priority for follow-up treatment than the second patient group.

System 100 may include one or more machine learning models. A machine learning model may be trained to create a computational model of anatomy of the patient. For example, a machine learning model may be trained using image data (CT data, fluoroscopy data, other imaging data, etc.), FFR data, and/or the like, to create the computational model. The machine learning model may also be trained, for example, using image data, FFR data, and/or the like, to generate a revised computational model of anatomy of the patient, which may represent the anatomy of the patient with certain anatomy changed, for example, a lesion removed, which may affect the flow stresses within the anatomy.

Computing device 150 may include, for example, an off-the-shelf device such as a laptop computer, desktop computer, tablet computer, smart phone, or other similar device or may include a specific purpose device. Computing device 150 may perform various control functions with respect to imager 140. In some examples, computing device 150 may include a guidance workstation. Computing device 150 may control the operation of imager 140 and receive the output of imager 140.

Display device 110 may be configured to output instructions, images, and messages relating to the medical procedure. Table 120 may be, for example, an operating table or other table suitable for use during a medical procedure.

In the example of FIG. 1, imager 140, such as a fluoroscopy imager, an angiography imager, or other imaging device, may be used to image relevant portions of the patient's anatomy during the medical procedure to visualize the anatomy, characteristics and locations of lesions or other issues inside the patient's body through the generation of imaging data. While described primarily as a fluoroscopy imager, imager 140 may be any type of imaging device, such as an angiography device, an intravascular ultrasound IVUS device, an OCT—FFR device, a CT device, an MRI device, a PET device, an ultrasound device, or the like. In some examples, imager 140 may represent more than one imaging device, such as a plurality of any of the aforementioned devices.

Imager 140 may image a region of interest in the patient's body. The particular region of interest may be dependent on anatomy, the medical procedure, patient symptoms, and/or the like. For example, when performing a cardiac medical procedure, a portion of the vasculature and/or the heart may be within the region of interest.

Computing device 150 may be communicatively coupled to imager 140, display device 110 and/or server 160, for example, by wired, optical, or wireless communications. Server 160 may be a hospital server which may or may not be located in an emergency room or Cath lab of a hospital, a cloud-based server, or the like. Server 160 may be configured to store patient imaging data, electronic healthcare or medical records or the like. Computing device 150 and/or server 160 may utilize image data from imager 140 and/or other data generated by devices of system 100, to generate a computational model of vasculature of a patient. For example, imager 140 may generate image data of the vasculature of the patient during a diagnostic medical procedure and/or during a therapeutic medical procedure. Computing device 150 and/or server 160 may use such image data to generate a computational model, such as a 3D model, of the vasculature of the patient.

Any of, or any combination of, computing device 150, imager 140, and/or server 160 may include a first computational model of vasculature of a first patient. For example, computing device 150, imager 140, and/or server 160 may determine a first culprit lesion based on the first computational model of the vasculature of the first patient. Computing device 150, imager 140, and/or server 160 may virtually remove the first culprit lesion from the first computational model to generate a revised first computational model. For example, this revised first computational model may be similar to the first computational model, but without the first culprit lesion being represented in the first computational model. With the first culprit lesion removed, blood flow within the vascular may change causing different pressures than with the first culprit lesion present. As such, computing device 150, imager 140, and/or server 160 may determine a first shear stress along first arterial walls in a first arterial tree based on the revised first computational model. Computing device 150, imager 140, and/or server 160 may compare the first shear stress to at least one threshold. Computing device 150, imager 140, and/or server 160 may determine that the first shear stress meets the at least one threshold in at least one location within the first arterial tree. For example, the at least one threshold may be relatively high enough to indicate that shear stress at the particular location of the revised first computational model may be higher than desired. As used herein, meeting a threshold may include being greater than the threshold, being greater than or equal to the threshold, being less than the threshold, or being less than or equal to the threshold, depending on the threshold itself and/or the usage of the threshold.

Computing device 150, imager 140, and/or server 160 may, based on the first shear stress meeting the at least one threshold, identify the patient as a member of a first patient group for follow-up treatment. For example, computing device 150, imager 140, and/or server 160 may identify the patient as being a relatively high-risk patient who should be prioritized for follow-up, such as further treatment or more intensive monitoring, over patients for whom the shear stress may not meet the threshold. As such, further testing and/or treatment may allow a clinician to more wholistically address cardiac issues of the patient and/or lower the risk to the patient of actually removing the first lesion without addressing the likely shear stress to be caused by the removal of the first lesion.

In some examples, in addition to, or alternatively, computing device 150, imager 140, and/or server 160 may reassess the group for follow-up with which patient is associated. For example, the prediction of the post-treatment state may be incorrect where a lesion was not removed or otherwise treated (e.g., bypassed) in a same manner as it was virtually removed prior to the actual, physical treatment. In some examples, computing device 150, imager 140, and/or server 160 may determine whether treatment for the first culprit lesion was applied as predicted (e.g., the same as or substantially the same as the virtual removal) and whether additional imaging data is desirable or a clinician may so indicate via a user interface. When additional imaging data is obtained or desired to be obtained, computing device 150 may obtain the additional image data and determine, based on the additional image data, whether the patient should remain a member of the first patient group for follow-up.

For example, computing device 150 may generate a post treatment computational model based on the additional image data from imager 140 after one or more lesions are actually treated. However, this may require a collection of a new set of image data which may expose the patient to additional X-rays and contrast. While such an example, may expose the patient to additional X-rays and contrast, it may be beneficial if an actual post-treatment state does not sufficiently match the pre-treatment prediction of the treatment effect and therefore pre-treatment prediction of future state may be in doubt. Computing device 150 may then undertake a similar analysis of shear stresses as described above to determine whether the patient should remain a member of the first patient group or be assigned to another patient group.

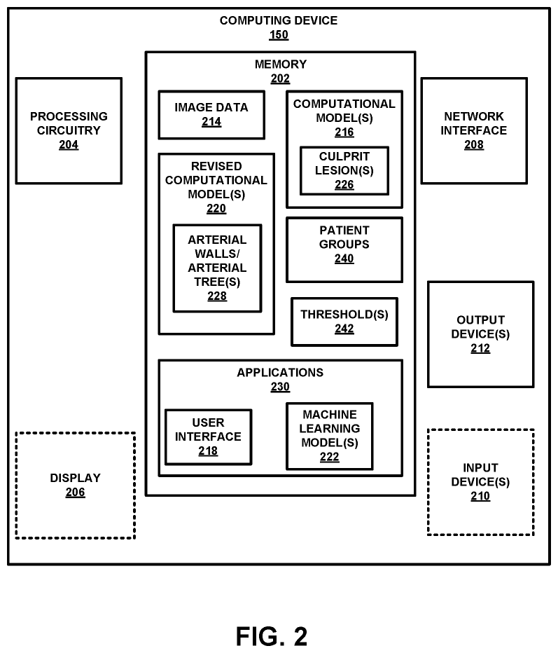

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with imager 140. As shown in FIG. 2, computing device 150 may represent multiple instances of computing devices, each of which may be associated with imager 140. Computing device 150 may include, for example, a memory 202, processing circuitry 204, a display 206, a network interface 208, input device(s) 210, and/or output device(s) 212, each of which may represent any of multiple instances of such a device within the computing system, for ease of description.

While processing circuitry 204 appears in computing device 150 in FIG. 2, in some examples, features attributed to processing circuitry 204 may be performed by processing circuitry of any of computing device 150, imager 140, or server 160, or combinations thereof. In some examples, one or more processors associated with processing circuitry 204 in computing system may be distributed and shared across any combination of computing device 150, imager 140, and server 160. Computing device 150 may be used to perform any of the techniques described in this disclosure, and may form all or part of devices or systems configured to perform such techniques, alone or in conjunction with other components, such as components of computing device 150, imager 140, server 160, or a system including any or all of such systems.

Memory 202 of computing device 150 includes any non-transitory computer-readable storage media for storing data or software that is executable by processing circuitry 204 and that controls the operation of computing device 150 and/or imager 140, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store image data 214, computational model(s) 216, revised computational model(s) 220, patient groups 240, and threshold(s) 242, and applications 230. Image data 214 may be generated by imager 140 of anatomy of the patient and obtained by computing device 150 via network interface 208 which may be communicatively coupled to imager 140. In some examples, image data 214 may include image data of anatomy of interest of the patient, such as cardiac vasculature of the patient. Memory 202 may store computational model 216. For example, processing circuitry 204 may generate computational model 216 based on image data 214. In some examples, computational model 216 may include a 3D model of anatomy of the patient.

Image data 214 may be captured by imager 140 (FIG. 1) during a medical procedure of a patient. Processing circuitry 204 may obtain imaging data 214 from imager 140 and store image data 214 in memory 202. Processing circuitry 204 may execute user interface 218 so as to cause display 206 (and/or display device 110 of FIG. 1) to present user interface 218 to one or more clinicians performing the medical procedure.

Applications 230 of memory 202 may include one or more machine learning model(s) 222 and user interface 218. Machine learning model(s) 222 may be configured to, when executed by processing circuitry 204, generate computational model(s) 216 based on image data 214 and/or generate revised computational model(s) 220. In some examples, processing circuitry 204 may determine a culprit lesion (e.g., of culprit lesion(s) 226) within the anatomy of the patient. For example, processing circuitry 204 may identify a culprit lesion, by determining that an FFR value at a particular location of the vasculature of the patient meets (e.g., is less than or less than or equal to) an FFR threshold. This may be indicative of a culprit lesion existing at that particular location. As such, processing circuitry 204 may identify the culprit lesion within a computational model of computational model(s) 216 as being at a location within the computational model that corresponds to the location within the vasculature of the patient having the FFR value that meets the threshold.

Processing circuitry 204 may virtually remove culprit lesion 226 from computational model 216 to generate a revised computational model (for example, of revised computational model(s) 220). Processing circuitry 204 may determine a shear stress along arterial walls in an arterial tree (e.g., of arterial walls/arterial tree(s) 228) based on a revised computational model of revised computational model(s) 220. For example, processing circuitry 204 may determine a velocity gradient at a vessel wall (e.g., an arterial wall of arterial walls/arterial tree 228).

Processing circuitry 204 may compare the shear stress to a threshold (for example, of threshold(s) 242). If the shear stress meets the threshold (e.g., is greater than or greater than or equal to), processing circuitry 204 may identify the patient as a member of a first patient group (e.g., of higher risk patients) for follow-up, such as further treatment or more intensive monitoring. If the shear stress does not meet the threshold, processing circuitry 204 may identify the patient as a member of a second patient group which may be a group of lower risk patients. Processing circuitry 204 may assign this second patient group of lower risk patients a lower priority for follow-up than the first patient group. For example, follow-up may include treatment and/or monitoring such as an incremental stress test, a CT scan, an angiography, or other technique to better investigate or treat the location(s) where the shear stress meets the threshold.

In some examples, there may be more than two patient groups. For example, there may be a plurality of first patient groups, each being associated with a shear stress meeting the threshold at a different particular location of the vasculature. For example, there may be a first patient group for patients whose shear stress meets the threshold at a carina. There may be another first patient group for patient whose shear stress meets the threshold within, for example, 5 mm of a carina. There may be another first patient group for patient whose shear stress meets the threshold at a side-branch bifurcation. There may be another first patient group for patient whose shear stress meets the threshold within, for example, 5 mm of a side-branch bifurcation. In this manner, the different first patient groups may be treated differently to schedule follow-up treatments more relevant for the areas of the vasculature for which the shear stress met the threshold. In some examples, there may be a plurality of thresholds, for example, one for each location in the vasculature.

In some examples, processing circuitry 204 may automatically schedule a patient for follow-up when the patient is identified as a member of the first patient group, but not automatically schedule another patient for follow-up treatment when that other patient is a member of the second patient group. In some examples, processing circuitry 204 may flag patient for follow-up treatment when the patient is identified as a member of the first patient group, but not flag another patient for follow-up treatment when that other patient is a member of the second patient group. The identity of such flagged patients may be sent to hospital staff responsible for scheduling follow-up appointments and the hospital staff may contact the flagged patients to schedule the follow-up appointments. In this manner, computing device 150 may prioritize the scheduling of follow-up medical procedures for the first group of relatively higher risk patients.

In some examples, processing circuitry 204 may create computational model 216 based on image data 214 (e.g., fluoroscopy data). For example, processing circuitry 204 may use multiplane fluoroscopy image data 214 to generate computational model 216. In some examples, computational model 216 may include a 3D model of anatomy of the patient.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated (e.g., via one or more sensors which may include one or more microphones), enabling display 206 to serve as both an input and output device. Alternatively, a keyboard (not shown), mouse (not shown), or other data input devices (e.g., input device(s) 210) may be employed.

Network interface 208 may be adapted to connect to a network such as a local area network (LAN) that includes a wired network or a wireless network, a wide area network (WAN), a wireless mobile network, a Bluetooth network, or the internet. For example, computing device 150 may obtain image data 214 from imager 140 during a medical procedure. Computing device 150 may receive updates to its software, for example, applications 230, via network interface 208. Computing device 150 may also display notifications on display 206 that a software update is available.

Input device(s) 210 may include any device that enables a user to interact with computing device 150, such as, for example, a mouse, keyboard, foot pedal, touch screen, augmented-reality input device receiving inputs such as hand gestures or body movements, or voice interface.

Output device(s) 212 may include any connectivity port or bus, such as, for example, parallel ports, serial ports, universal serial busses (USB), or any other similar connectivity port known to those skilled in the art.

Applications 230 may be one or more software programs stored in memory 202 and executed by processing circuitry 204 of computing device 150. Processing circuitry 204 may execute user interface 218, which may display image data 214, representations of computational model 216, revised computational model 220, culprit lesion 226, arterial walls/arterial tree 228 on display 206 and/or display device 110.

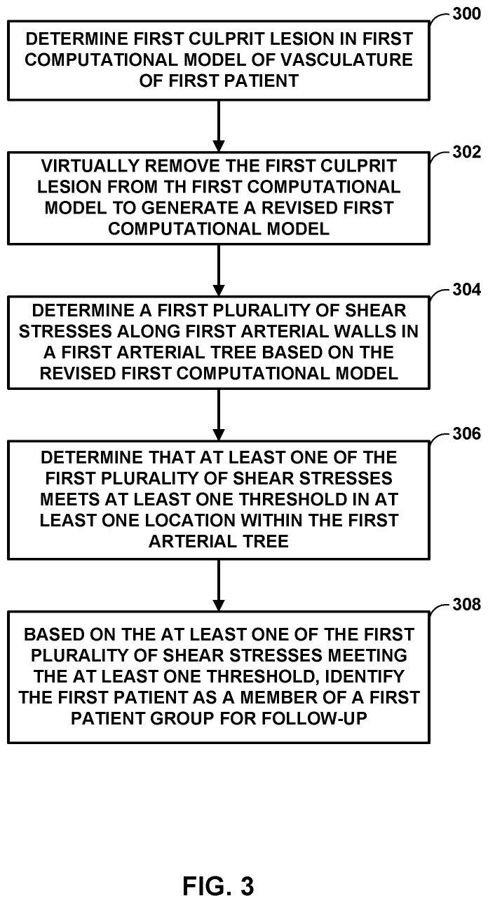

FIG. 3 is a flow diagram of example techniques for identifying patients for follow-up according to one or more aspects of this disclosure. The techniques of FIG. 3 are described below with respect to processing circuitry 204, but such techniques may be performed by any of, or any combination of, processing circuitry of devices depicted in FIG. 1 or capable of performing such techniques.

Processing circuitry 204 may determine a first culprit lesion in the first computational model of the vasculature of the first patient (300). For example, processing circuitry 204 may determine an FFR value meets an FFR threshold. The FFR value at a particular location meeting the FFR threshold, may indicate a culprit lesion exists at that location. The location in the computational model associated with the location of the patient having the FFR value meeting the FFR threshold may be identified as including culprit lesion 226.

Processing circuitry 204 may virtually remove the first culprit lesion from the first computational model to generate a revised first computational model (302). For example, processing circuitry 204 may remove culprit lesion 226 from a computational model of computational model(s) 216 to generate a revised computational model of computational model(s) 220.

Processing circuitry 204 may determine a first plurality of shear stresses along first arterial walls in a first arterial tree based on the revised first computational model (304). For example, processing circuitry 204 may determine a shear stress map including a plurality of shear stress values along arterial walls/arterial tree(s) 228.

Processing circuitry 204 may determine that at least one of the first plurality of shear stresses meets at least one threshold in at least one location within the first arterial tree (306). For example, processing circuitry 204 may compare the values of a shear stress map to a threshold associated with a location within arterial walls/arterial tree(s) 228 to determine that at least one of the first plurality of shear stresses meets at least one threshold in at least one location within the first arterial tree.

Processing circuitry 204 may, based on the at least one of the first plurality of shear stresses meeting the at least one threshold, identify the first patient as a member of a first patient group for follow-up (308). For example, processing circuitry 204 may, based on at least one value of the shear stress map meeting the at least one threshold, identify the first patient as a member of a first patient group of patient groups 240 for follow-up, such as treatment and/or monitoring.

In some examples, processing circuitry 204 is further configured to determine a second culprit lesion (e.g., of culprit lesions 226) based on a second computational model (e.g., of computational model(s) 216) of a vasculature of a second patient. In such examples, processing circuitry 204 is further configured to virtually remove the second culprit lesion from the second computational model to generate a revised second computational model (e.g., of revised computational models 220). In such examples, processing circuitry 204 is configured to determine a second plurality of shear stresses along arterial walls in a second arterial tree (e.g., of arterial walls/arterial tree(s) 228) based on the revised second computational model. In such examples, processing circuitry 204 is configured to determine that none of the second plurality of shear stresses meet the at least one threshold (e.g., of threshold(s) 242) in at least one location within the second arterial tree. In such examples, processing circuitry 204 is configured to, based on none of the second plurality of shear stresses meeting the at least one threshold, identify the second patient as a member of a second patient group (e.g., of patient groups 240), wherein the second patient group comprises a lower risk patient group than the first patient group. In some examples, processing circuitry 204 is further configured to prioritize, for follow-up, the first patient group over the second patient group.

In some examples, the follow-up includes at least one of an incremental stress test, CT scan, or angiography. In some examples, as part of determining the first culprit lesion, processing circuitry 204 is configured to determine that an FFR value meets an FFR threshold.

In some examples, the at least one location comprises a predetermined location. In some examples, the predetermined location comprises at least one of at a carina, within 5 mm of a carina, at a side-branch bifurcation, or within 5 mm of a side-branch bifurcation.

In some examples, as part of determining the shear stress, processing circuitry 204 is configured to determine a velocity gradient at a vessel wall. In some examples, the at least one threshold includes a plurality of thresholds each associated with a respective location within the arterial tree.

In some examples, processing circuitry 204 is further configured to, based on the patient being identified as a member of the first patient group, at least one of flagging the patient for follow-up or automatically scheduling the patient for follow-up. In some examples, processing circuitry 204 is further configured to obtain image data of the first patient vasculature and determine the first computational model of the first patient vasculature based on the image data.

In some examples, processing circuitry 204 may obtain additional image data of the first patient vasculature (e.g., of image data 214). The additional image data may be captured by an imaging device (e.g., imager 140) after a treatment for the first culprit lesion is performed, for example, after the first culprit lesion is removed, bypassed, or the like. Processing circuitry 204 may determine, based on the additional image data, whether the first patient should remain a member of the first patient group.

In some examples, as part of determining whether the first patient should remain a member of the first patient group, processing circuitry 204 may be configured to determine a post treatment computational model (e.g., of computational model(s) 216), the post treatment computational model being based at least in part on the additional image data.

In some examples, processing circuitry 204 is further configured to determine a post treatment plurality of shear stresses along the first arterial walls in the first arterial tree based on the post treatment computational model. In such examples, processing circuitry 204 is configured to determine that at least one of the post treatment plurality of shear stresses meets the at least one threshold in at least one location within the first arterial tree. In some examples, processing circuitry 204 is configured to, based on the at least one of the post treatment plurality of shear stresses meeting the at least one threshold, determine that the first patient remain a member of the first patient group. In some examples, processing circuitry 204 is configured to, based on the at least one of the post treatment plurality of shear stresses not meeting the at least one threshold, determine that the first patient be removed from the first patient group and be identified as member of the second patient group.

In some examples, processing circuitry 204 is further configured to determine a difference between the treatment for the first culprit lesion and the virtual removal of the first culprit lesion. For example, processing circuitry 204 may determine that not all of the first culprit lesion which was removed virtually was actually physically removed or that the first culprit lesion was not actually removed at all, for example, that the first culprit lesion was bypassed. Processing circuitry 204 may determine that the difference meets at least one criterion (e.g., of threshold(s) 242). For example, the at least one criterion may include a threshold percentage of actual removal compared to the virtual removal, that the treatment was different than an attempted removal, or the like. Based on the difference meeting the at least one criterion, processing circuitry 204 may generate an indication suggesting that the additional imaging data be obtained. For example, processing circuitry 204 may output the suggestion to display 206 and/or display device 110, or send a communication to a clinician computing device (not shown) including the indication.

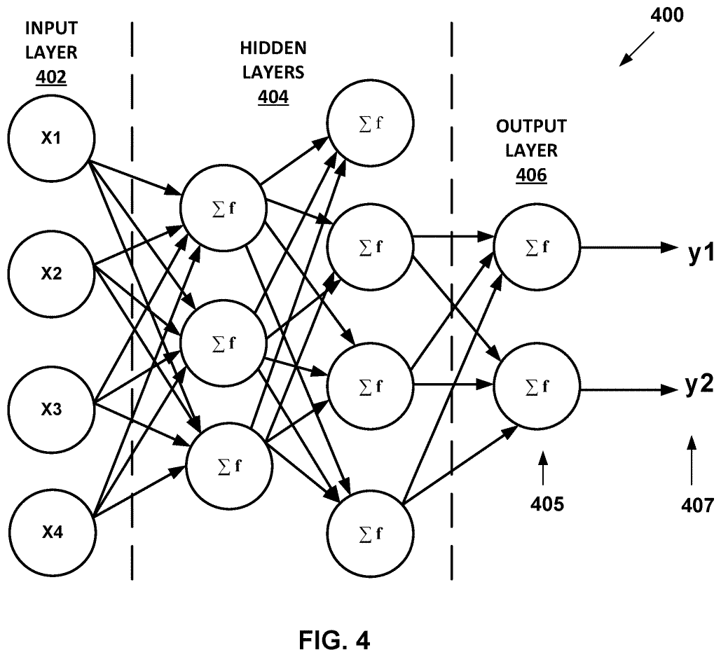

FIG. 4 is a conceptual diagram illustrating an example machine learning model according to one or more aspects of this disclosure. Machine learning model 400 may be an example of machine learning model(s) 222. Machine learning model 400 may be an example of a deep learning model, or deep learning algorithm, trained to generate computational model(s) 216 and/or revised computational model(s) 220. One or more of computing device 150 and/or server 160 may train, store, and/or utilize machine learning model 400, but other devices of system 100 may apply inputs to machine learning model 400 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 4, machine learning model 400 may include three types of layers. These three types of layers include input layer 402, hidden layers 404, and output layer 406. Output layer 406 comprises the output from the transfer function 405 of output layer 406. Input layer 402 represents each of the input values X1 through X4 provided to machine learning model 400. In some examples, the input values may include any of the values input into the machine learning model, as described above. For example, the input values may include image data 214, as described above. In addition, in some examples input values of machine learning model 400 may include additional data, such as other data that may be collected by or stored in system 100.

Each of the input values for each node in the input layer 402 is provided to each node of a first layer of hidden layers 404. In the example of FIG. 4, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between image data 214 and computational model(s) 216 and/or revised computational model(s) 220. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be a classification that image data 214 is indicative of aspects of a computational model of computational model(s) 216, culprit lesion(s) 226, revised computational model(s) 220, and/or arterial walls/arterial tree(s) 228.

As shown in the example above, by applying machine learning model 400 to input data such as image data 214, processing circuitry 204 is able to determine a particular computational model and/or revised computational model. This may improve the placement of a patient into an appropriate patient group, which may improve patient outcomes while more efficiently using medical resources.

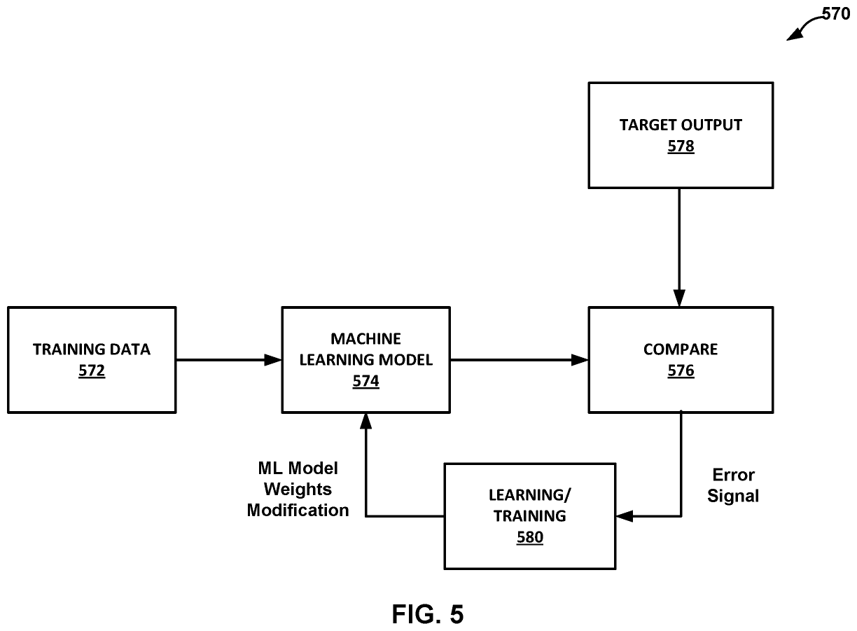

FIG. 5 is a conceptual diagram illustrating an example training process for a machine learning model according to one or more aspects of this disclosure. Process 570 may be used to train machine learning model(s) 222 or machine learning model 400. A machine learning model 574 (which may be an example of machine learning model 400 and/or machine learning model(s) 222) may be implemented using any number of models for supervised and/or reinforcement learning, such as but not limited to, an artificial neural network, a decision tree, naïve Bayes network, support vector machine, or k-nearest neighbor model, CNN, RNN, LSTM, ensemble network, to name only a few examples. In some examples, one or more of computing device 150 and/or server 160 initially trains machine learning model 574 based on a corpus of training data 572. Training data 572 may include, for example, image data (CT data, fluoroscopy data, etc.) of anatomy of patients, FFR data, sensor data (e.g., from a wearable device, implantable device, stethoscope, etc.), and/or patient metadata (e.g., sex, age, weight, height, body mass index, body fat percentage, comorbidities, cholesterol level, blood pressure, blood oxygenation, physical exercise level, heart rate, or the like). Training data 572 may include data from past medical procedures performed on a plurality of patients having the same, similar, and/or different patient conditions, prior medical procedures, and/or the like. In some examples, processing circuitry 204 may execute machine learning model(s) 222 to generate computational model(s) 216 and/or revised computational model(s) 220.

While training machine learning model 574, processing circuitry of system 2 may compare 576 a prediction or classification with a target output 578. Processing circuitry 204 may utilize an error signal from the comparison to train (learning/training 580) machine learning model 574. Processing circuitry 204 may generate machine learning model weights or other modifications which processing circuitry 204 may use to modify machine learning model 574. For examples, processing circuitry 204 may modify the weights of machine learning model 400 based on the learning/training 580. For example, one or more of computing device 150 and/or server 160, may, for each training instance in training data 572, modify, based on training data 572, the manner in which a computational model and/or revised computational is determined.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.
Example 1. A medical system comprising: memory configured to store a first computational model of a first patient vasculature; and processing circuitry communicatively coupled to the memory, the processing circuitry being configured to: determine a first culprit lesion based on the first computational model of the vasculature of the first patient; virtually remove the first culprit lesion from the first computational model to generate a revised first computational model; determine a first plurality of shear stresses along first arterial walls in a first arterial tree based on the revised first computational model; determine that at least one of the first plurality of shear stresses meets at least one threshold in at least one location within the first arterial tree; and based on the at least one of the first plurality of shear stresses meeting the at least one threshold, identify the first patient as a member of a first patient group for follow-up.
Example 2. The medical system of example 1, wherein the processing circuitry is further configured to: determine a second culprit lesion based on a second computational model of a vasculature of a second patient; virtually remove the second culprit lesion from the second computational model to generate a revised second computational model; determine a second plurality of shear stresses along arterial walls in a second arterial tree based on the revised second computational model; determine that none of the second plurality of shear stresses meet the at least one threshold in at least one location within the second arterial tree; and based on none of the second plurality of shear stresses meeting the at least one threshold, identify the second patient as a member of a second patient group, wherein the second patient group comprises a lower risk patient group than the first patient group.
Example 3. The medical system of example 2, wherein the processing circuitry is further configured to prioritize, for follow-up, the first patient group over the second patient group.
Example 4. The medical system of any of examples 1-3, wherein the follow-up comprises at least one of an incremental stress test, CT scan, or angiography.
Example 5. The medical system of any of examples 1-4, wherein as part of determining the first culprit lesion, the processing circuitry is configured to determine a fractional flow reserve (FFR) value meets an FFR threshold.
Example 6. The medical system of any of examples 1-5, wherein the at least one location comprises a predetermined location.
Example 7. The medical system of example 6, wherein the predetermined location comprises at least one of at a carina, within 5 mm of a carina, at a side-branch bifurcation, or within 5 mm of a side-branch bifurcation.
Example 8. The medical system of any of examples 1-7, wherein as part of determining the first plurality of shear stresses, the processing circuitry is configured to determine a velocity gradient at a vessel wall.
Example 9. The medical system of any of examples 1-8, wherein the at least one threshold comprises a plurality of thresholds each associated with a respective location within the arterial tree.
Example 10. The medical system of any of examples 1-9, wherein the processing circuitry is further configured to, based on the first patient being identified as a member of the first patient group, at least one of flagging the first patient for follow-up or automatically scheduling the first patient for follow-up.
Example 11. The medical system of any of examples 1-10, wherein the processing circuitry is further configured to: obtain image data of the first patient vasculature; and determine the first computational model of the first patient vasculature based on the image data.
Example 12. The medical system of any of examples 1-11, wherein the processing circuitry is further configured to: obtain additional image data of the first patient vasculature, the additional image data being captured by an imaging device after a treatment for the first culprit lesion is performed; and determine, based on the additional image data, whether the first patient should remain a member of the first patient group.
Example 13. The medical system of example 12, wherein as part of determining whether the first patient should remain a member of the first patient group, the processing circuitry is configured to determine a post treatment computational model, the post treatment computational model being based at least in part on the additional image data.
Example 14. The medical system of example 13, wherein the processing circuitry is further configured to: determine a post treatment plurality of shear stresses along the first arterial walls in the first arterial tree based on the post treatment computational model; determine that at least one of the post treatment plurality of shear stresses meets the at least one threshold in at least one location within the first arterial tree; and based on the at least one of the post treatment plurality of shear stresses meeting the at least one threshold, determine that the first patient remain a member of the first patient group.
Example 15. The medical system of example 13, wherein the processing circuitry is further configured to: determine a post treatment plurality of shear stresses along the first arterial walls in the first arterial tree based on the post treatment computational model; determine that at least one of the post treatment plurality of shear stresses meets the at least one threshold in at least one location within the first arterial tree; and based on the at least one of the post treatment plurality of shear stresses not meeting the at least one threshold, determine that the first patient be removed from the first patient group and be identified as member of the second patient group.
Example 16. The medical system of any of examples 12-15, wherein the processing circuitry is further configured to: determine a difference between the treatment for the first culprit lesion and the virtual removal of the first culprit lesion; determine that the difference meets at least one criterion; and based on the difference meeting the at least one criterion, generate an indication suggesting that the additional imaging data be obtained.
Example 17. A method comprising: determining, by processing circuitry, a first culprit lesion based on a first computational model of vasculature of a first patient; virtually removing, by the processing circuitry, the first culprit lesion from the first computational model to generate a revised first computational model; determining, by the processing circuitry, a first plurality of shear stresses along first arterial walls in a first arterial tree based on the revised first computational model; determining, by the processing circuitry, that at least one of the first plurality of shear stresses meets at least one threshold in at least one location within the first arterial tree; and based on the at least one of the first plurality of shear stresses meeting the at least one threshold, identifying, by the processing circuitry, the first patient as a member of a first patient group for follow-up.
Example 18. The method of example 17, wherein the processing circuitry is further configured to: determining, by processing circuitry, a second culprit lesion based on a second computational model of a vasculature of a second patient; virtually removing, by processing circuitry, the second culprit lesion from the second computational model to generate a revised second computational model; determining, by processing circuitry, a second plurality of shear stresses along arterial walls in a second arterial tree based on the revised second computational model; determining, by processing circuitry, that none of the second plurality of shear stresses meet the at least one threshold in at least one location within the second arterial tree; and based on none of the second plurality of shear stresses meeting the at least one threshold, identifying, by processing circuitry, the second patient as a member of a second patient group, wherein the second patient group comprises a lower risk patient group than the first patient group.
Example 19. The method of example 18, further comprising prioritizing, by the processing circuitry and for follow-up, the first patient group over the second patient group.
Example 20. The method of any of examples 17-19, wherein the follow-up comprises at least one of an incremental stress test, CT scan, or angiography.
Example 21. The method of any of examples 17-20, wherein determining the first culprit lesion comprises determining that an FFR value falls below an FFR threshold.
Example 22. The method of any of examples 17-21, wherein the at least one location comprises a predetermined location.
Example 23. The method of example 22, wherein the predetermined location comprises at least one of at a carina, within 5 mm of a carina, at a side-branch bifurcation, or within 5 mm of a side-branch bifurcation.
Example 24. The method of any of examples 17-23, wherein determining the first plurality of shear stresses comprises determining a velocity gradient at a vessel wall.
Example 25. The method of any of examples 17-24, wherein the at least one threshold comprises a plurality of thresholds each associated with a respective location within the arterial tree.
Example 26. The method of any of examples 17-25, further comprising, based on the first patient being identified as a member of the first patient group, at least one of flagging the first patient for follow-up or automatically scheduling the first patient for follow-up.
Example 27. The method of any of examples 17-26, further comprising: obtaining, by the processing circuitry, image data of the first patient vasculature; and determining, by the processing circuitry, the first computational model of the first patient vasculature based on the image data.
Example 28. A non-transitory computer-readable storage medium storing instructions, which when executed cause processing circuitry to: determine a first culprit lesion based on a first computational model of vasculature of a first patient; virtually remove the first culprit lesion from the first computational model to generate a revised first computational model; determine a first plurality of shear stresses along first arterial walls in a first arterial tree based on the revised first computational model; determine that at least one of the first plurality of shear stresses meets at least one threshold in at least one location within the first arterial tree; and based on the at least one of the first plurality of shear stresses meeting the at least one threshold, identify the first patient as a member of a first patient group for follow-up.

Various examples have been described. These and other examples are within the scope of the following claims.

1. A medical system for determining a stratification of a patient for follow-up comprising:
memory configured to store a first computational model of a first patient vasculature; and
processing circuitry communicatively coupled to the memory, the processing circuitry being configured to:
determine a first culprit lesion based on the first computational model of the vasculature of the first patient;
virtually remove the first culprit lesion from the first computational model to generate a revised first computational model;
determine a first plurality of shear stresses along first arterial walls in a first arterial tree based on the revised first computational model;
determine that at least one of the first plurality of shear stresses meets at least one threshold in at least one location within the first arterial tree; and
based on the at least one of the first plurality of shear stresses meeting the at least one threshold, identify the first patient as a member of a first patient group for follow-up.

2. The medical system of claim 1, wherein the processing circuitry is further configured to:
determine a second culprit lesion based on a second computational model of a vasculature of a second patient;
virtually remove the second culprit lesion from the second computational model to generate a revised second computational model;
determine a second plurality of shear stresses along arterial walls in a second arterial tree based on the revised second computational model;
determine that none of the second plurality of shear stresses meet the at least one threshold in at least one location within the second arterial tree; and
based on none of the second plurality of shear stresses meeting the at least one threshold, identify the second patient as a member of a second patient group, wherein the second patient group comprises a lower risk patient group than the first patient group.

3. The medical system of claim 2, wherein the processing circuitry is further configured to prioritize, for follow-up, the first patient group over the second patient group.

4. The medical system of claim 1, wherein the follow-up comprises at least one of an incremental stress test, CT scan, or angiography.

5. The medical system of claim 1, wherein as part of determining the first culprit lesion, the processing circuitry is configured to determine an FFR value meets an FFR threshold.

6. The medical system of claim 1, wherein the at least one location comprises a predetermined location.

7. The medical system of claim 6, wherein the predetermined location comprises at least one of at a carina, within 5 mm of a carina, at a side-branch bifurcation, or within 5 mm of a side-branch bifurcation.

8. The medical system of claim 1, wherein as part of determining the shear stress, the processing circuitry is configured to determine a velocity gradient at a vessel wall.

9. The medical system of claim 1, wherein the at least one threshold comprises a plurality of thresholds each associated with a respective location within the arterial tree.

10. The medical system of claim 1, wherein the processing circuitry is further configured to, based on the first patient being identified as a member of the first patient group, at least one of flagging the first patient for follow-up or automatically scheduling the first patient for follow-up.

11. The medical system of claim 1, wherein the processing circuitry is further configured to:
obtain image data of the first patient vasculature; and
determine the first computational model of the first patient vasculature based on the image data.

12. The medical system of claim 1, wherein the processing circuitry is further configured to:
obtain additional image data of the first patient vasculature, the additional image data being captured by an imaging device after a treatment for the first culprit lesion is performed; and
determine, based on the additional image data, whether the first patient should remain a member of the first patient group.

13. The medical system of claim 12, wherein the processing circuitry is further configured to:
determine a difference between the treatment for the first culprit lesion and the virtual removal of the first culprit lesion;
determine that the difference meets at least one criterion; and
based on the difference meeting the at least one criterion, generate an indication suggesting that the additional imaging data be obtained.

14. A method for determining a stratification of a patient for follow-up comprising:
determining, by processing circuitry, a first culprit lesion based on a first computational model of vasculature of a first patient;
virtually removing, by the processing circuitry, the first culprit lesion from the first computational model to generate a revised first computational model;
determining, by the processing circuitry, a first plurality of shear stresses along first arterial walls in a first arterial tree based on the revised first computational model;
determining, by the processing circuitry, that at least one of the first plurality of shear stresses meets at least one threshold in at least one location within the first arterial tree; and
based on the at least one of the first plurality of shear stresses meeting the at least one threshold, identifying, by the processing circuitry, the first patient as a member of a first patient group for follow-up.

15. A non-transitory computer-readable storage medium storing instructions, which when executed cause processing circuitry to determine a stratification of a patient for follow-up, wherein the instructions cause the processing circuitry to:
determine a first culprit lesion based on a first computational model of vasculature of a first patient;
virtually remove the first culprit lesion from the first computational model to generate a revised first computational model;
determine a first plurality of shear stresses along first arterial walls in a first arterial tree based on the revised first computational model;
determine that at least one of the first plurality of shear stresses meets at least one threshold in at least one location within the first arterial tree; and
based on the at least one of the first plurality of shear stresses meeting the at least one threshold, identify the first patient as a member of a first patient group for follow-up.

16. The medical system of claim 13, wherein the processing circuitry is further configured to:
determine a post treatment plurality of shear stresses along the first arterial walls in the first arterial tree based on the post treatment computational model;
determine that at least one of the post treatment plurality of shear stresses meets the at least one threshold in at least one location within the first arterial tree; and
based on the at least one of the post treatment plurality of shear stresses meeting the at least one threshold, determine that the first patient remain a member of the first patient group.

17. The medical system of claim 12, wherein the processing circuitry is further configured to:
determine a post treatment plurality of shear stresses along the first arterial walls in the first arterial tree based on the post treatment computational model;
determine that at least one of the post treatment plurality of shear stresses meets the at least one threshold in at least one location within the first arterial tree; and
based on the at least one of the post treatment plurality of shear stresses not meeting the at least one threshold, determine that the first patient be removed from the first patient group and be identified as member of the second patient group.

18. The medical system of claim 12, wherein the processing circuitry is further configured to:
determine a difference between the treatment for the first culprit lesion and the virtual removal of the first culprit lesion;
determine that the difference meets at least one criterion; and
based on the difference meeting the at least one criterion, generate an indication suggesting that the additional imaging data be obtained.

19. The method of claim 14, further comprising:
determining, by the processing circuitry, a second culprit lesion based on a second computational model of a vasculature of a second patient;
virtually removing, by the processing circuitry, the second culprit lesion from the second computational model to generate a revised second computational model;
determining, by the processing circuitry, a second plurality of shear stresses along arterial walls in a second arterial tree based on the revised second computational model;
determining, by the processing circuitry, that none of the second plurality of shear stresses meet the at least one threshold in at least one location within the second arterial tree; and
based on none of the second plurality of shear stresses meeting the at least one threshold, identifying, by the processing circuitry, the second patient as a member of a second patient group, wherein the second patient group comprises a lower risk patient group than the first patient group.

20. The method of claim 19, further comprising prioritizing, by the processing circuitry and for follow-up, the first patient group over the second patient group.
